(19) United States
(12) Patent Application Publication
(10) Pub. No.: US 2021/0130897 A1
(43) Pub. Date: May 6, 2021

(54) SALIVARY MICRORNA LEVELS IN ANOREXIA NERVOSA PROVIDE A LIQUID BIOPSY OF METABOLIC AND NEUROPSYCHIATRIC STATUS

(71) Applicants: QUADRANT BIOSCIENCES INC., Syracuse, NY (US); THE RESEARCH FOUNDATION FOR THE STATE UNIVERSITY OF NEW YORK, Syracuse, NY (US); PENN STATE RESEARCH FOUNDATION, University Park, PA (US)

(72) Inventors: Steven D. HICKS, Hershey, PA (US); Frank A. MIDDLETON, Fayetteville, NY (US); Richard UHLIG, Ithaca, NY (US)

(73) Assignees: QUADRANT BIOSCIENCES INC., Syracuse, NY (US); THE RESEARCH FOUNDATION FOR THE STATE UNIVERSITY OF NEW YORK, Syracuse, NY (US); PENN STATE RESEARCH FOUNDATION, University Park, PA (US)

(21) Appl. No.: 17/048,022

(22) PCT Filed: Apr. 15, 2019
(86) PCT No.: PCT/US2019/027510

§ 371 Date: Oct. 15, 2020

Related U.S. Application Data
(60) Provisional application No. 62/658,007, filed on Apr. 16, 2018.

(51) Int. Cl. C12Q 1/6883

(52) U.S. Cl. CPC C12Q 1/6883; C12Q 2600/178; C12Q 2600/158

(57) ABSTRACT

A method for detecting a risk of, diagnosing, prognosing, or monitoring anorexia nervosa or another eating disorder or for distinguishing a healthy subject from one with anorexia, an eating disorder, or an anxiety disorder comprising detecting at least one abnormal or altered pattern of miRNA and/or microbial RNA in biological sample compared to a control value from one or more normal subjects or healthy controls, and selecting a subject having at least one abnormal or altered pattern of miRNA and/or microbial RNA correlated with anorexia nervosa or another eating disorder. Probe and/or primer compositions for identifying miRNAs or microbial RNAs; compositions containing miRNAs or microbial RNAs, their mimics or agents that target miRNAs or microbial RNAs; and methods of treatment for AN and other eating disorders.

### BACKGROUND

### Field of the Invention

MicroRNA biomarkers for anorexia nervosa and other eating disorders and their symptoms.

### Description of the Related Art

Eating disorders (“EDs”) are among the leading causes of morbidity and mortality in young females of high-income countries, causing greater disease burden than common conditions such as alcohol use disorders or gynecological disorders.¹ Anorexia nervosa (“AN”) is an ED with high rates of chronicity, morbidity and mortality,² with the highest mortality rate of any psychiatric disorder.³

The etiology of AN is unknown and it remains unclear whether the underlying primary disturbance is of pathways that control appetite or if these symptoms are secondary to other disturbances such as anxiety or obsessions around weight gain.³

As many as two-thirds of AN patients suffer from co-morbid anxiety symptoms; see Kaye, W. H., Bulik, C. M Thornton, L., Barbarich, N., Masters, K., & Price Foundation Collaborative Group. (2004). Comorbidity of anxiety disorders with anorexia and bulimia nervosa. American journal of psychiatry, 161(12), 2215-2221.

Recent studies have indicated that 50-80% of the risk for developing an ED can be attributed to genetic heritability which emphasizes the neurobiological underpinnings of AN.² In addition, much of the current research has focused on understanding neurocircuits that may underlie the relationship between feeding-weight behavior and reward-emotional responses to appetitive stimuli seen in AN.³ Because of the increasing evidence of altered neurobiology underlying the onset of EDs, biomarkers may be of use in understanding the etiology of AN as well as predicting treatment course and outcome.

As the molecular pathophysiology of AN is not well understood, treatments for the primary symptoms are lacking. Additionally, there is currently no accurate tool for predicting treatment course for patients suffering from AN. Current management guidelines rely on subjective assessment tools that establish a DSM-5 diagnosis but do not greatly inform prognosis.⁴ In the widest-scale review of outcome of AN to date, there was a mortality rate of about 5.0%, while on average, full recovery was found in only less than half of all patients.⁵ Of the remaining patients, 33% improved, and 20% developed a chronic course of the disorder. Because of the variable course of treatment and outcomes seen in patients with AN, a tool capable of predicting outcomes could be extremely beneficial for clinicians.

The use of peripheral micro-ribonucleic acid (“miRNA”) profiles as biomarkers for human disease is an emerging trend in medicine,⁷ however, to our knowledge, no study has explored this potential in AN. MiRNAs are small, non-coding, epi-transcriptional molecules that regulate protein production. Precursor miRNAs are cleaved from a stem-loop configuration by the RNA-induced silencing (“RISC”) complex to form active single-stranded mature molecules, which silence coding mRNAs through targeted, complementary binding; see Gregory, R. I., Chendrirnada, T. P., Cooch, N., & Shiekhattar, R. (2005). Human RISC couples mic oRNA biogenesis and posttranscriptional gene silencing. Cell, 123(4). 631-640. Functionality of the RISC complex and down-stream miRNA production are critical for a number of physiologic processes, including nervous system development and host metabolism; see Fiore, R., Siegel, G., & Schratt. G. (2008). MicroRNA function in neuronal development, plastic and disease. Biochimica et Biophysics Acta (BBA)-Gene Regulatory Mechanisms, 1779(8), 471-478; Lynn, F. C. (2009). Meta-regulation: microRNA regulation of glucose and 1 id metabolism Trends in Endocrinology & Metabolism, 20(9), 452-459.

Preliminary studies show that miRNA is altered in patients with psychiatric disorders that are often co-morbid with AN, such as depression and anxiety,⁹⁻¹¹ and that salivary miRNA is altered by disorders of the nervous system.¹² These studies indicate that peripheral miRNA patterns may be used as an accurate biomarker for differentiating patients with and without some CNS disorders, but whether similar patterns can be found in patients with AN remains to be seen. Given the disrupted nutritional and neuropsychiatric states that occur in patient with AN, saliva, would seem a particularly suitable biofluid for interrogation.

In view of the above, the inventors investigated whether salivary miRNA profiles would be disrupted in subjects having AN relative to healthy peers and whether such miRNA disruptions differ based on prandial status (i.e., fasting or non-fasting status). They also investigated while controlling for co-morbid AX symptoms whether comparison of miRNA levels among AN and anxiety (“AX”) patients might specifically yield miRNAs affected by a state of chronic malnutrition.

### SUMMARY OF THE INVENTION

A method for detecting a risk of, diagnosing, prognosing, or monitoring anorexia nervosa or another eating disorder or for distinguishing a healthy subject from one with anorexia, an eating disorder, or an anxiety disorder comprising detecting at least one abnormal or altered pattern of miRNA and/or microbial RNA in biological sample compared to a control value from one or more normal subjects or healthy controls, and selecting a subject having at least one abnormal or altered pattern of miRNA and/or microbial RNA correlated with anorexia nervosa or another eating disorder. Probe and/or primer compositions for identifying miRNAs or microbial RNAs; compositions containing miRNAs or microbial RNAs, their mimics or agents that target miRNAs or microbial RNAs; and methods of treatment for AN and other eating disorders.

### BRIEF DESCRIPTION OF THE DRAWINGS

FIG. 1A. Global salivary miRNA profiles partially distinguish AN patients. A two-dimensional partial least squares discriminant analysis (PLSDA) utilizing the 353 most robustly expressed salivary miRNAs (raw counts of 10 or more in at least 33% of samples) accounted for 17.3% of the variance in the data while spatially separating AN samples (red X) from HC (green circles) and AX (blue triangles) controls. Note that fasting samples for all groups (denoted with “f”) are more centrally clustered, while post-prandial samples (denoted with “p”) display peripheral dispersion. Shaded areas represent 95% confidence interval for each group.

FIG. 1B. Eight miRNAs demonstrated variable importance projection (VIP) scores ≥2.0 for the PLSDA. Four were mature miRNAs (denoted with “hsa-”) and four were precursor miRNAs (denoted with “pre-”). Five miRNAs demonstrated relative up-regulation (red boxes) in AN samples, relative to HC and AX controls. Two miRNAs were down-regulated in AN samples (green boxes), and one miRNA had intermediary expression in AN saliva (yellow box).

FIG. 1C/Supplemental FIG. 1B. ASCA AN meal status interaction.

FIG. 2. Hierarchical clustering of AN samples using expression levels of 8 salivary miRNAs. The 58 saliva samples collected from anorexia (AN, n=20), healthy control (HC, n=18), and anxiety (AX, n=20) participants were subjected to hierarchical clustering, using the expression levels from the top eight miRNAs identified with PLSDA analysis. AN samples (red) generally segregated from AX (blue) and HC (green) samples. Notably, only 5/20 AN samples were displaced from the group cluster, and 4/5 were post-prandial samples. A Pearson distance metric with complete clustering algorithm distributed the eight miRNAs into three groups, including three miRNAs generally down-regulated in AN samples (hsa-miR-378i, pre-miR-145, pre-miR-744) and three miRNAs generally up-regulated in AN samples (hsa-miR-203a-3p, pre-miR-200c, hsa-miR-200b-3p).

FIG. 3. Three salivary miRNAs with unique responses to caloric intake in AN patients. A comparison of fasting (n=10) and post-prandial (n=10) samples from 10 AN participants with fasting (n=18) and post-prandial (n=20) samples from 18 HC and 12 AX controls was performed with a 2-way ANOVA. There were three salivary miRNAs with nominal interactions (p<0.05) between AN-status and meal-status. Boxplots of pre-miR-15b (p=0.019), hsa-let-7f-2-3p (p=0.006), and pre-miR-629 (p=0.029) are shown.

FIGS. 4A, 4B and 4C.

### DETAILED DESCRIPTION OF THE INVENTION

Micro ribonucleic acids (miRNAs) regulate protein translation, influencing processes like metabolism and brain function, but have not been studied in anorexia nervosa (AN). The purpose of this study was to identify potential diagnostic and therapeutic salivary miRNAs “altered” in AN.

Five individual miRNAs demonstrated between group differences on SAM and two of these miRNAs (miR-200c, and hsa-miR-203a-3p) had significant VIP scores (≥2.0) for clustering AN, AX, and HC samples. PLSDA and hierarchical clustering analyses revealed distinct distribution patterns for pre- and post-prandial samples, and three miRNAs were found to have nominal interactions (p<0.05) between AN-status and prandial-status on 2-way ANOVA (pre-miR-15b, pre-miR-629, hsa-let-7f-2-3p). The miRNAs identified on SAM, PLSDA, and 2-way ANOVA showed target enrichment for genes involved in fatty acid biosynthesis (p=2.06E-18, 4 genes, 2 miRNAs) and fatty acid metabolism (p=4.00E-7, 14 genes, 8 miRNAs). Individual miRNA levels were associated with both medical characteristics and neuropsychiatric measures.

As shown herein and in the Example below, the inventors found that salivary miRNA profiles are disrupted in adolescent females with AN and that these miRNAs are influenced by prandial-status, target genes related to fatty acid metabolism, and are related to neuropsychiatric measures. Such miRNA markers provide an objective measure for tracking AN severity or treatment response.

Saliva is a slightly alkaline secretion of water, mucin, protein, salts, and often a starch-splitting enzyme (as ptyalin) that is secreted into the mouth by salivary glands, lubricates ingested food, and often begins the breakdown of starches. Saliva is released by the submandibular gland, parotid gland, and/or sublingual glands and saliva release may be stimulated by the sympathetic and/or parasympathetic nervous system activity. Saliva released primarily by sympathetic or parasympathetic induction may be used to isolate microRNAs.

Saliva may be collected by expectoration, swabbing the mouth, passive drool, or by other methods known in the art. It can be collected from the mouth prior to or after a rinse. For example, in some embodiments it may be collected without rinsing the mouth first and in other embodiments after rinsing accumulated saliva out of the mouth and collecting newly secreted saliva, optionally after the administration of a sialagogue, such as a parasympathomimetic drug (e.g., pilocarpine) acting on parasympathetic muscarinic receptors, such as the M3 receptor, to induce an increased saliva flow. Malic acid, ascorbic acid, chewing gum or plant or herbal extracts that promote saliva flow may also be used. In other embodiments saliva may be withdrawn from a salivary gland.

In some embodiments, a saliva sample may be further purified by centrifugation, filtration, or other means that preserves miRNA content. For example, it may be filtered through a 0.22 micron or 0.45 micron membrane and the separated components, such as cells, microvesicles, or fluids used to recover microRNAs or microbial RNAs.

In other embodiments, proteins or enzymes that degrade microRNA may be removed, inactivated or neutralized in a saliva sample, for example, a RNAse inhibitor such as Superase In RNase Inhibitor, may be added to a sample containing miRNA.

MicroRNA or miRNA is a small non-coding RNA molecule containing about 22 nucleotides, which is found in plants, animals and some viruses, that functions in RNA silencing and post-transcriptional regulation of gene expression; see Ambros, V (Sep. 16, 2004). The functions of animal microRNAs. Nature. 431 (7006): 350-5. doi:10.1038/nature02871. PMID 15372042; or Bartel, D P (Jan. 23, 2004). MicroRNAs: genomics, biogenesis, mechanism, and function. Cell. 116 (2): 281-97. doi:10. 1016/S0092-8674(04)00045-5. PMID 14744438, both of which are incorporated by reference.

A miRNA standard nomenclature system uses the prefix “miR” followed by a dash and a number, the latter often indicating order of naming. For example, miR-120 was named and likely discovered prior to miR-241. A capitalized “miR-” refers to the mature form of the miRNA, while the uncapitalized “mir-” refers to the pre-miRNA and the pri-miRNA, and “MIR” refers to the gene that encodes them. Mature miRNAs herein are often denoted with “hsa-” and precursor miRNAs denoted with “pre-”.

Microbial RNA is RNA produced by microbes such as those present in the oral cavity. It may be collected from saliva by procedures similar to those described above for miRNA.

miRNA or microbial RNA isolation from biological samples such as saliva and their analysis may be performed by methods known in the art, including the methods described by Yoshizawa, et al., Salivary MicroRNAs and Oral Cancer Detection, Methods Mol Biol. 2013; 936: 313-324; doi: 10.1007/978-1-62703-083-0 (incorporated by reference) or by using commercially available kits, such as mirVana™ miRNA Isolation Kit which is incorporated by reference to the literature available at https://_tools.thermofisher.com/content/sfs/manuals/fm_1560.pdf (last accessed Jan. 30, 2018).

Mimics. In some embodiments, miRNA mimics may be employed. Such mimics may be small, double-stranded RNA molecules designed to mimic endogenous mature miRNA molecules once transfected into a cell. Mimics may target and modulate the expression of the same gene(s) as the corresponding native miRNA or may be designed to have lower, higher, or altered activity on target gene(s). Mimics are often used for gene silencing. They generally contain a sequence at least partially complementary to a three prime untranslated region (3′-UTR) of a target gene or sequence. A seed sequence that targets a miRNA to a particular RNA generally contains 6-8 nucleotides complementary to a target RNA sequence. A mimic may comprise the same seed sequence as a miRNA described herein.

Some miRNA mimics may contain non-natural nucleotides. Artificial nucleic acids such as locked nucleic acids (“LNAs”) or bridged nucleic acids (“BNAs”) may be used as mimics. Such mimics are commercially available; see http://_www.biosyn.com/bna-synthesis-bridged-nucleic-acid.aspx (last accessed Jan. 22, 2018, incorporated by reference).

Such miRNA mimics may be designed based on information available in the miRBase; http://_www.mirbase.org/ (ver. 21) (last accessed Jan. 22, 2018) which is incorporated by reference. In other embodiments a partial or full complement of an miRNA or an miRNA mimic may be designed. Such complements will bind to a target miRNA.

Next Generation Sequencing refers to non-Sanger-based high-throughput DNA sequencing technologies. Millions or billions of DNA strands can be sequenced in parallel, yielding substantially more throughput and minimizing the need for the fragment-cloning methods that are often used in Sanger sequencing of genomes. Next generation sequencing methods useful for sequencing miRNA and microbial RNAs are known and incorporated by reference to https://_en.wikipedia.org/wiki/DNA sequencing (last accessed Jan. 30, 2018).

DIANA-mirPath is a miRNA pathway analysis web-server, providing accurate statistics, while being able to accommodate advanced pipelines. mirPath can utilize predicted miRNA targets (in CDS or 3′-UTR regions) provided by the DIANA-microT-CDS algorithm or even experimentally validated miRNA interactions derived from DIANA-TarBase. These interactions (predicted and/or validated) can be subsequently combined with sophisticated merging and meta-analysis algorithms; see Vlachos, Ioannis S., Konstantinos Zagganas, Maria D. Paraskevopoulou, Georgios Georgakilas, Dimitra Karagkouni, Thanasis Vergoulis, Theodore Dalamagas, and Artemis G. Hatzigeorgiou. DIANA-miRPath v3. 0: deciphering microRNA function with experimental support. Nucleic acids research (2015): gkv403 (incorporated by reference) and http://_snf-515788.vm.okeanos.grnet.gr/ (last accessed Jan. 25, 2018, incorporated by reference.

MicrobiomeAnalyst is software that provides comprehensive statistical, visual and meta-analysis of microbiome data; see http://_www.microbiomeanalyst.ca/faces/home.xhtml (incorporated by reference; last accessed Jan. 31, 2018).

MetaboAnalyst is a comprehensive tool for metabolomics analysis and interpretation; see http://_www.metaboanalyst.ca/ (incorporated by reference; last accessed Jan. 31, 2018).

Ingenuity® Pathway Analysis is an analysis and search tool that uncovers the significance of ‘omics data and identifies new targets or candidate biomarkers within the context of biological systems; see https://_www.qiagenbioinformatics.com/products/ingenuity-pathway-analysis/(incorporated by reference, last accessed Jan. 31, 2018).

Normalization. Data may be normalized to improve data quality or facilitate analysis or comparison. For example a subject's miRNA level may be normalized to a fasting level or to a level in a healthy control subject. A subject's epigenetic and/or microbiome genetic sequence data may be normalized to account for inter-sample count variations; such count normalization utilizing one or more invariant miRNAs or microbial RNAs so as to represent data in proportion to their relative expression. Normalization methods for RNA sequence data may also be used; see the methods described by Li, et al., BMC Informatics 16:347, Comparing the normalization methods for the differential analysis of Illumina high-throughput RNA-Seq data (2015; incorporated by reference).

### Example

Methods. This study was approved by the Institutional Review Board at the Penn State Milton S. Hershey College of Medicine. Written informed consent was provided by all participants. Participant assent was provided where appropriate.

Design. A prospective case-control design was used to compare salivary microRNA profiles among females (13-22 years of age) in three groups: 1) restrictive-type anorexia nervosa (AN), at time of enrollment into a partial hospitalization program (n=10); 2) age- and sex-matched controls with anxiety disorder (AX, n=12); and 3) age- and sex-matched healthy controls (HC) without anxiety or disordered eating (n=18). Given the high incidence of anxiety co-morbidity in adolescent females with AN, the AX comparator group was included to control for the potential influence of neuropsychiatric disturbances on peripheral miRNA patterns. In addition, AN and AX participants provided both fasting and post-prandial saliva samples to examine the influence of AN on the miRNA response to caloric intake.

Participants. Participants were females, age 11-21 years, with: 1) restrictive-type AN, at the outset of partial hospitalization treatment; 2) anxiety (AX); and 3) healthy controls (HC). AN participants included 10 females who met criteria on the Diagnostic and Statistics Manual (DSM)-5 criteria for restrictive AN undergoing admission to the Penn State Health Children's Hospital eating disorder partial hospitalization program.

The ANX group included 10 female adolescents meeting DSM-5 criteria for anxiety disorder (with or without co-morbid depression) presenting for care at the child and adolescent psychiatry outpatient clinic.

The HC group included 18 healthy college-age female distance runners. Exclusion criteria for all groups included a primary language other than English, periodontal disease, acute upper respiratory illness, ongoing neurologic disorder (e.g. seizures, intellectual disability), and drug/alcohol dependency.

HC and ANX participants with positive score on the Children's Eating Attitudes Test (ChEAT) or a history of eating disorder were excluded. HC participants with positive scores on the Patient Health Questionnaire (PHQ-9) or the Generalized Anxiety Disorder 7-item scale (GAD-7) were excluded.

Basic demographic (age, sex, race), anthropometric (height, weight, body mass index), and medical data (medical history, current medications) were collected for each participant. All AN participants completed Revised Children's Manifest Anxiety Scale (RCMAS), Children's Depression Inventory (CDI), Child Behavior Checklist (CBCL), and Eating Disorder Examination Questionnaire (EDEA) measures. In addition, duration of eating disorder symptoms, timing of last menses were recorded. Thyroid stimulating hormone, total T3, estradiol, pre-albumin, and heart rate were extracted from AN patients' medical records.

AN participants had a mean age of 14 (±1) years and were 100% Caucasian, with a mean BMI of 16±1 (% mBMI of 84±7). HC participants had a mean age of 19 (±1) years and were 94% Caucasian, with a mean BMI of 20±1 (% mBMI of 92±6). AX participants had a mean age of 16 (±2) years and were 75% Caucasian with a mean BMI of 24±6 (% mBMI of 118±30). AN participants were younger than HC (p=2.6E-12) and AX (p=0.012) participants. They also had lower % mBMI than HC (p=0.032) and AX (p=0.0014) participants.

There were 40% of AN participants with a co-morbid DSM-5 diagnosis of generalized anxiety disorder (GAD) and 30% with major depressive disorder (MDD). Forty percent of AN participants were taking a selective serotonin re-uptake inhibitor (SSRI). They had a mean score on the GAD7 of 14±6, and a mean score on the PHQ9 of 14±7. There were 66% of AX participants with GAD. The remaining 33% had a DSM-5 diagnosis of social anxiety disorder. Fifty-eight percent of AN participants had a co-morbid DSM-5 diagnosis of MDD, and 66% were taking a SSRI. AX participants had a mean GAD7 score of 10±5 (p=0.21) and a mean PHQ9 score of 10±6 (p=0.21).

There were no HC participants with a diagnosis of GAD or MDD, and no HC participants taking an SSRI. HC participants had a mean GAD7 score of 2±2 (p=2.3E-7) and a mean PHQ9 score of 3±3 (p=1.2E-5).

AN characteristics. Extensive neuropsychiatric and medical testing was performed on the AN group. On the RCMAS, AN participants had a mean score of 52 (±10). AN participants had a mean total score of 57 (±10) on the CBCL and a mean total score of 63 (±15) on the CDI. Their mean scores on the EDEQ were 77±27 (total), 1.4±0.6 (restraint), 0.8±0.4 (eating), 2.1±0.8 (shape), 1.1±0.5 (weight), and 1.5±0.4 (global). AN participants had a mean duration of eating disorder symptoms of 4 (±2) months, and had lost 22 (±8) percent of body weight, on average. They reported mean daily caloric intake of 680 (±340) Kcal/day, and a mean duration of 3 (±2) months since last menses. AN participants had a mean thyroid stimulating hormone (TSH) level of 2.3 (±1.1) U/ml, a mean total triiodothyronine (T3) level of 56 (±12) ng/dL, a mean pre-albumin of 20 (±3) pg/ml, and a mean resting heart rate of 49 (±6) beats per minute.

Saliva Collection. Saliva was collected from all participants through expectoration between 7 AM and 10 AM following an oral tap-water rinse. Oragene RNA RE-100 collection kits were employed (DNA Genotek, Ottawa, Canada). AN and AX participants provided paired fasting (no food intake for >8 hours) and post-prandial samples. Timing of last meal was recorded for all HC participants, who provided either a morning fasting (n=7), or post-prandial sample (n=11). Samples were briefly stored at room temperature prior to transfer to −20° C. and processing at the SUNY Molecular Analysis Core at Upstate Medical University (Syracuse, N.Y.).

RNA Processing. Salivary miRNA was purified using a Trizol technique with a second RNeasy mini column purification (Qiagen, Germantown, Md.). RNA yield and quality were checked with the Agilent Bioanalyzer before library construction. RNA was sequenced using an Illumina TruSeq Small RNA Sample Prep protocol (Illumina; San Diego, Calif.) and a NextSeq500 instrument (Illumina, San Diego, Calif.). RNA was interrogated at a targeted read depth of ten million reads per sample, using 50 base pair single end reads. Reads were aligned to the hg38 build of the human genome in Partek Flow (Partek; St. Louis, Mo.) with the SHRiMP2 aligner. Total miRNA counts within each sample were quantified with miRBase precursor and mature-microRNA v21. Of the 4469 miRNAs aligned, we interrogated only the 353 miRNAs with robust salivary expression (defined as raw counts of 10 or greater in at least 33% of samples). These parameters were chosen to capture any miRNAs that might be uniquely expressed in only one group of participants.

Quantification of miRNAs. As described above, morning pre- and post-prandial salivary samples were collected. RNA was quantified using high throughput sequencing. Partek Flow was used to align mature and premature miRNAs with the human genome. Significance analysis of microarray (SAM) identified individual miRNAs with differences between 20 AN, 20 AX and 18 HC samples. Variable importance projection (VIP) scores were used to quantify miRNA contributions to group separation on partial least squares discriminant analysis (PLSDA), and hierarchical clustering was used to visualize expression patterns across individual samples. Fasting and post-prandial miRNA levels were compared across AN and non-AN samples with 2-way analysis of variance (ANOVA). MiRNAs of interest were functionally interrogated in DIANA miRPath software. Relationships between miRNAs and medical/neuropsychiatric characteristics were investigated with Pearson correlation analyses.

Statistical Analysis. Differences in individual miRNA expression across AN, AX, and HC groups were investigated with a non-parametric Kruskal-Wallis analysis of variance (ANOVA). A significance analysis of microarray (SAM) technique was used to distinguish those miRNAs with significant between-group differences (delta=0.65, FDR≤0.15). Global miRNA profiles were visualized across the three groups with a two-dimensional partial least squares discriminant analysis (PLSDA). Variable importance in projection (VIP) scores were determined for individual miRNAs and those with VIP≥2.0 were reported. The miRNAs with integral to PLSDA projection were used to perform hierarchical clustering of individual saliva samples with a Pearson's distance metric and a complete clustering algorithm.

To interrogate interactions between AN-status and caloric intake, all saliva samples were designated as: 1) AN, or non-AN; 2) fasting, or post-prandial. An ANOVA Simultaneous Components Analysis (SCA) was used to explore potential interactions between meal- and AN-status on salivary miRNA expression. Differences in individual miRNAs across groups were identified with a 2-way ANOVA. The relationship between miRNAs of interest (those identified on ANOVA or VIP analysis) and medical/demographic variables was explored with Pearson correlation testing. Relationships between miRNA levels and the neuropsychiatric measures or medical variables collected in AN participants were also explored with Pearson's analysis.

The potential functional impact of salivary miRNA expression patterns in AN was investigated through down-stream pathway analysis of predicted mRNA targets. Gene targets were identified for three groups of salivary miRNAs: 1) those “altered” in AN versus AX samples (predicted nutritional miRNAs); 2) those “altered” in AN versus HC samples (predicted neuropsychiatric miRNAs); and 3) those with AN-meal interactions (potentially defining the unique AN response to caloric intake). High confidence gene targets (targetscan context score ≥−0.3) for miRNAs of interest were identified in DIANA miRPath software, and enrichment of KEGG ontology pathways was identified with Fisher's Exact Testing with false detection rate correction (FDR <0.05). Expression levels for mRNA targets of the miRNAs of interest were interrogated post-hoc through alignment of RNA sequencing outputs to the coding human transcripts database in Partek Flow. Alignment and RNA filtering was completed as described for miRNAs above and comparison of salivary mRNA levels across groups was investigated with non-parametric Kruskal-Wallis ANOVA. Correlations in salivary concentration between “altered” miRNA-mRNA target pairs were investigated with a Pearson correlation analysis.

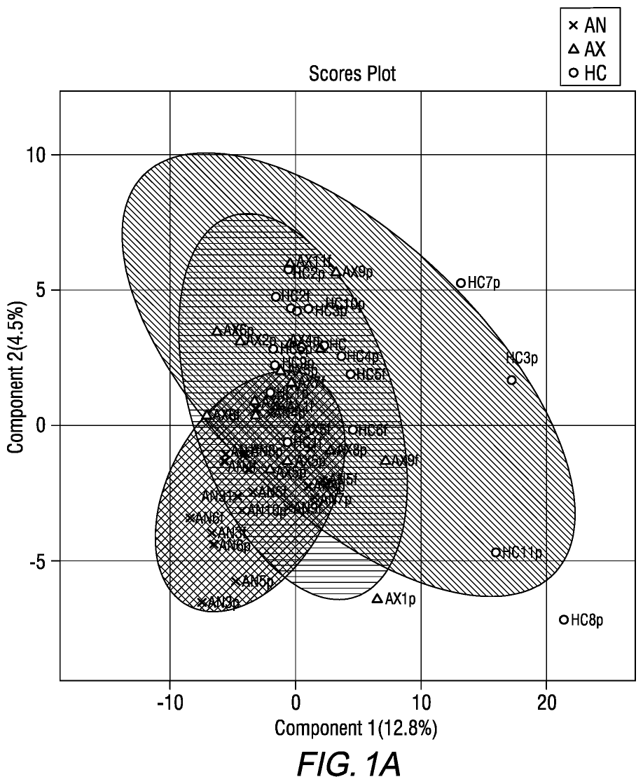

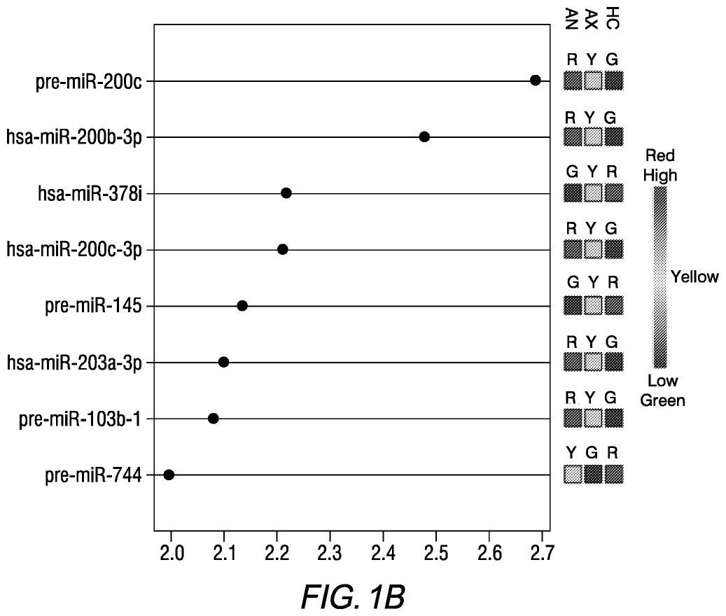

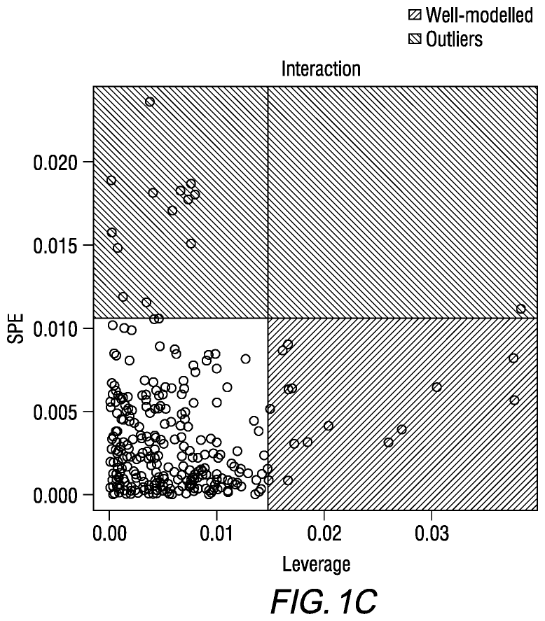

Salivary miRNA profiles. Visualization of global miRNA expression across AN, AX, and HC groups with two-dimensional PLSDA resulted in partial separation of groups, while accounting for 17.3% of variance in the miRNA dataset (FIG. 1A). Interestingly, fasting samples tended to be centrally clustered with peripheral dispersion of post-prandial samples. There were eight miRNAs with a variable importance projection (VIP) score ≥2.0 (FIG. 1B). Five of these miRNAs (pre-miR-200c, hsa-miR-200b-3p, hsa-miR-200c-3p, hsa-miR-203a-3p, and pre-miR-103b-1) demonstrated the lowest expression in AN samples, two miRNAs had the highest expression in AN samples (hsa-miR-378i, and pre-miR-145), and one miRNA had intermediate expression in AN samples (pre-miR-744). Significance analysis of microarray (delta=0.65) identified five individual miRNAs with significant differences between groups (Table 1).

TABLE 1
Significant salivary miRNAs on SAM

| MicroRNA | d.value | stdev | rawp | q.value | KW chi.squared |
|---|---|---|---|---|---|
| pre-mir-200c | 3.082 | 0.827 | 0.001 | 0.154 | 9.553 |
| hsa-miR-30d-5p | 2.935 | 0.837 | 0.002 | 0.154 | 10.812 |
| hsa-miR-374a-3p | 2.769 | 0.847 | 0.002 | 0.154 | 10.131 |
| pre-mir-30a | 2.610 | 0.857 | 0.003 | 0.154 | 9.708 |
| hsa-miR-200b-3p | 2.557 | 0.860 | 0.003 | 0.154 | 7.736 |

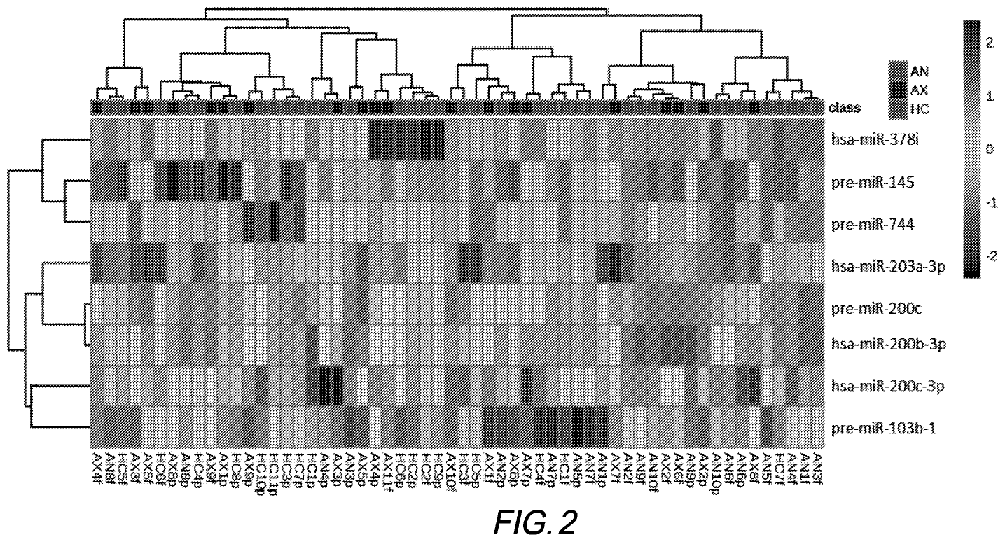

Two of these miRNAs (miR-200c, and hsa-miR-203a-3p) were among the eight VIP candidates. Hierarchical clustering of AN, AX, and HC samples using a Pearson distance metric with a complete clustering algorithm for the 8 VIP miRNAs generally segregated AN from HC and AX samples (FIG. 2). Interestingly, 3/5 mis-clustered AN samples were from patients with co-morbid generalized anxiety. In addition, mis-clustered AN samples tended to be post-prandial (4/5 mis-clustered samples), while mis-clustered AX and HC samples tended to be fasting (6/7 mis-clustered samples).

The potential relationship observed between AN-status and meal-status on hierarchical clustering (FIG. 2) and PLSDA (FIG. 1A), was explored further in a two-way ANOVA. ANOVA-SCA utilizing global salivary miRNA expression demonstrated significant model validation (p≤0.05) for AN-status (AN versus HC/AX) and meal-status (fasting versus post-prandial), but not AN-meal interactions (FIG. 1B). This model identified 18 miRNAs with significant (leverage threshold >0.9; alpha <0.05) predictive ability for AN-meal interactions (FIG. 1C/Supplemental FIG. 1B). Of the 18 miRNAs six were nominally (p<0.05) affected by AN-status on 2-way ANOVA, seven were affected by meal-status, and three demonstrated a meal-AN interaction (Table 2).

TABLE 2
Significant salivary miRNAs on ASCA

| | | | 2-way | 2-way | Meal-AN |
|---|---|---|---|---|---|
| | | | ANOVA | ANOVA | status |
| | Lever- | | AN-status | Meal-status | Interaction |
| MicroRNA | age | SPE | (pval) | (pval) | (pval) |
| hsa-let-7a-5p | 0.038 | 0.006 | 0.019 | 0.006 | 0.056 |
| hsa-miR-200b-3p | 0.038 | 0.008 | 0.002 | 0.004 | 0.087 |
| hsa-let-7f-2-3p | 0.030 | 0.006 | 0.531 | 0.423 | 0.006 |
| pre-mir-629 | 0.027 | 0.004 | 0.947 | 0.141 | 0.029 |
| pre-mir-98 | 0.026 | 0.003 | 0.109 | 0.020 | 0.129 |
| pre-mir-15b | 0.020 | 0.004 | 0.095 | 0.611 | 0.019 |
| hsa-miR-629-5p | 0.018 | 0.003 | 0.678 | 0.401 | 0.053 |
| hsa-miR-6073 | 0.017 | 0.003 | 0.855 | 0.060 | 0.148 |
| hsa-miR-197-3p | 0.017 | 0.006 | 0.497 | 0.074 | 0.097 |
| pre-mir-197 | 0.017 | 0.006 | 0.443 | 0.103 | 0.088 |
| pre-mir-29a | 0.017 | 0.001 | 0.024 | 0.003 | 0.729 |
| hsa-miR-203a-3p | 0.017 | 0.009 | 0.005 | 0.121 | 0.365 |
| hsa-miR-106a-5p | 0.016 | 0.009 | 0.888 | 0.001 | 0.060 |
| pre-mir-103b-1 | 0.015 | 0.005 | 0.002 | 0.015 | 0.487 |
| pre-mir-374c | 0.015 | 0.001 | 0.025 | 0.005 | 0.603 |

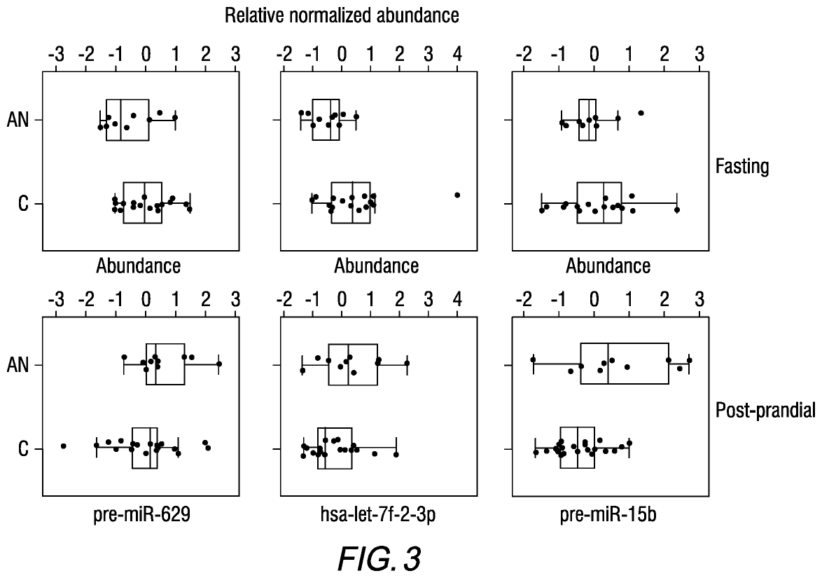

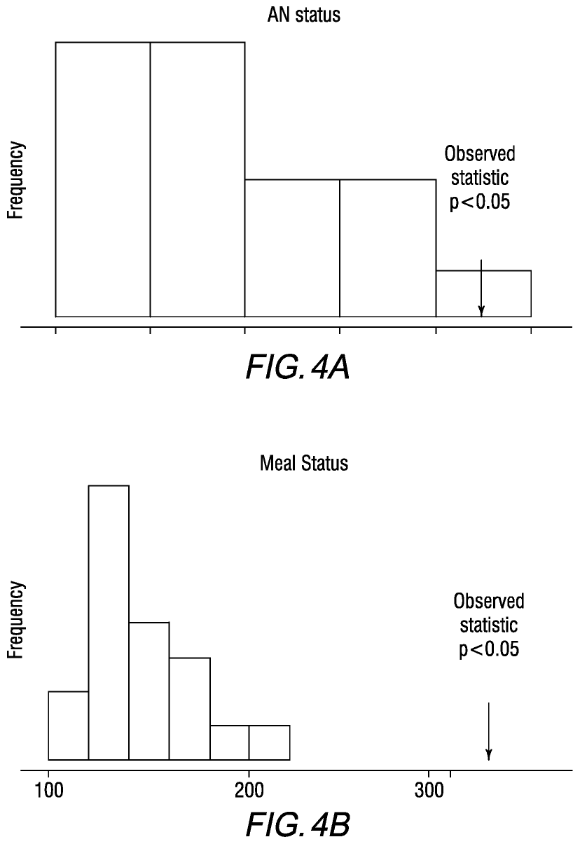

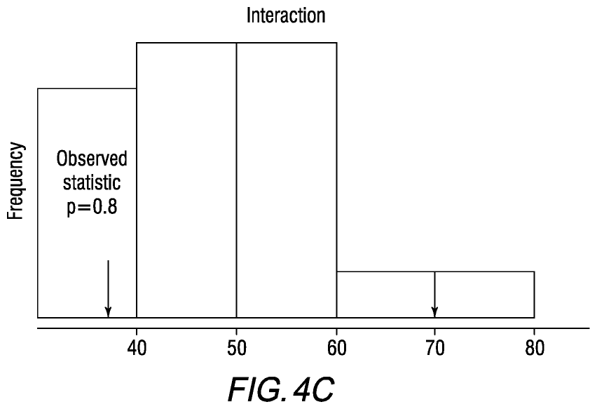

Three of these 18 miRNAs were among the 8 VIP miRNA candidates separating AN, AX, and HC samples on PLSDA (hsa-miR-200b-3p, hsa-miR-203a-3p, pre-miR-103b-1). The three miRNAs with significant meal-AN interactions, tended to be lower in fasting AN samples compared with fasting control samples, but higher in AN samples post-meal (FIG. 3).

The 14 salivary miRNAs identified on SAM, PLSDA, and 2-way ANOVA were explored for associations with 15 medical factors related to AN-status using a Pearson correlation analysis. The 14 miRNAs demonstrated 36 significant (p<0.05) associations with AN-related measures (Table 3).

TABLE 3
Medical features associated with fasting, post-prandial,
and meal-induced changes in salivary miRNA levels

| | Fasting miRNA | Post-prandial | Post-meal miRNA |
|---|---|---|---|
| MicroRNA | level | miRNA level | change |
| Pre-miR-200c | Estradiol (0.70, | ED duration | Estradiol (−0.73, |
| | 0.024) | (−0.88, 7.2E−4) | 0.017) |
| Hsa-miR-30d-5p | — | HR (0.74, 0.013), | HR (0.72, 0.018) |
| | | GAD7 (−0.70, 0.024), | |
| | | SSRI use (−0.70, | |
| | | 0.024), RCMAS | |
| | | (−0.66, 0.038) | |
| Hsa-miR-374a-3p | T3 (−0.74, 0.014), | PHQ9 (−0.67, 0.035) | — |
| | CDI (−0.70, 0.023), | | |
| | EBW (−0.67, 0.034), | | |
| | GAD7 (−0.63, 0.049) | | |
| Pre-miR-30a | | Weeks since menses | Weeks since menses |
| | | (−0.7, 0.023) | (−0.67, 0.032) |
| Hsa-miR-200b-3p | Estradiol (0.68, | ED duration (−0.83, | Estradiol (−0.81, |
| | 0.030) | 0.003) | 0.004) |
| Pre-miR-15b | CBCL (−0.74, 0.015), | Estradiol (−0.75, | Estradiol (−0.74, |
| | TSH (0.70, 0.024) | 0.011) | 0.015) |
| Hsa-let-7f-2-3p | — | GAD7 (−0.63, 0.050) | — |
| Pre-miR-629 | — | EDEQ Eating (−0.71, | EDEQ Weight (−0.64, |
| | | 0.021) | 0.045) |
| Hsa-miR-378i | TSH (−0.75, 0.007) | BW loss (0.65, | — |
| | | 0.043), TSH (−0.77, | |
| | | 0.009) | |
| Hsa-miR-200c-3p | — | TSH (−0.72, 0.019); | — |
| | | CBCL (0.65, 0.041) | |
| Pre-miR-145 | — | — | Estradiol (−0.80, |
| | | | 0.006) |
| Hsa-miR-203a-3p | — | — | — |
| Pre-miR-103b-l | EDEQ Eating (−0.73, | — | — |
| | 0.017), TSH (−0.65, | | |
| | 0.043), EDEQ Total | | |
| | (−0.63, 0.049) | | |
| Pre-miR-744 | ED duration (−0.66, | — | ED duration (0.64, |
| | 0.039) | | 0.044) |

*Associations between miRNA concentrations and medical factors determined with Pearson Correlation analysis. R-value and p-values are displayed for each medical factor listed.
Abbreviations: Thyroid stimulating hormone level (TSH), Eating disorder examination questionnaire (EDEQ), eating disorder (ED), Child behavior checklist total (CBCL), Generalized anxiety disorder 7-item scale (GAD7), Children's depression inventory (CDI), expected body weight (EBW), Revised children's manifest anxiety scale score (RCMAS), Patient health questionnaire-9 score (PHQ9), resting heart rate (HR), selective serotonin re-uptake inhibitor use (SSRI), body weight (BW) loss.

Estradiol level was associated with 4/14 miRNAs (pre-miR-200c, hsa-miR-200b, pre-miR-15b, pre-miR-145). There were also three miRNAs (hsa-miR-103b, hsa-miR-378i, miR-200c) associated with thyroid stimulating hormone level. The strongest relationship was an inverse association between pre-miR-200c and duration of eating disorder (R=−0.88, p=7.2E-4)). Notably, pre-miR-629 displayed negative associations with both the eating (R=−0.71, p=0.021) and weight (R=−0.64, p=0.045) components of the eating disorder examination questionnaire. There were also several associations between levels of hsa-miR-30d-5p and indices of patient anxiety (GAD7 score, RCMAS score, SSRI use). Relationships between mean expression for the 14 miRNAs of interest and body mass index were explored for all participants (AN, AX and HC). Body mass index demonstrated a direct association with salivary levels of hsa-let-7f-2-3p (R=0.39, p=0.008) and an inverse association with pre-miR-103b-1 (R=−0.33, p=0.027). Levels of hsa-let-7f-2-3p were also directly associated with % mBMI (R=0.42, p=0.003).

The 14 miRNAs of interest had 4124 potential gene targets (targetscan context score >0.3). These targets demonstrated enrichment (FDR<0.05) for 18 KEGG pathways (Table 4A). The two pathways with the most significant enrichment were fatty acid biosynthesis (p=2.06E-18, 4 genes, 2 miRNAs) and fatty acid metabolism (p=4.00E-7, 14 genes, 8 miRNAs). There were six other enriched pathways involved in metabolism (biotin metabolism, p=3.66E-5) or biosynthetic processes (steroid hormone biosynthesis, p=9.43E-6; glycosaminoglycan biosynthesis, p=0.000784; valine/leucine/isoleucine biosynthesis, p=0.00187; mucin type 0-glycan biosynthesis, p=0.020; and N-glycan biosynthesis, p=0.0173). Targeting of metabolic and biosynthetic pathways was particularly pronounced when target enrichment was performed for the 5/8 VIP miRNAs with elevated levels in AN participants (Table 4B).

TABLE 4A
AN-related salivary miRNAs target genes involved in metabolic and
biosynthetic pathways

| KEGG pathway | p-value | #genes | #miRNAs |
|---|---|---|---|
| Fatty acid biosynthesis | 2.06E−18 | 4 | 2 |
| Fatty acid metabolism | 4.00E−07 | 14 | 8 |
| Signaling pathways | 3.83E−06 | 45 | 14 |
| regulating pluripotency | | | |
| of stem cells | | | |
| Steroid hormone | 9.43E−06 | 16 | 8 |
| biosynthesis | | | |
| Biotin metabolism | 3.66E−05 | 2 | 2 |
| Proteoglycans in cancer | 0.000376 | 55 | 13 |
| Renal cell carcinoma | 0.000617 | 26 | 10 |
| Glycosaminoglycan | 0.000784 | 9 | 9 |
| biosynthesis - heparan | | | |
| sulfate/heparin | | | |
| Wnt signaling pathway | 0.00099 | 42 | 14 |
| Valine, leucine and | 0.001875 | 3 | 2 |
| isoleucine biosynthesis | | | |
| Basal cell carcinoma | 0.005103 | 22 | 12 |
| TGF-beta signaling pathway | 0.007458 | 22 | 10 |
| Hedgehog signaling pathway | 0.01079 | 20 | 10 |
| N-Glycan biosynthesis | 0.017352 | 13 | 9 |
| Mucin type O-Glycan | 0.020016 | 8 | 8 |
| biosynthesis | | | |
| Colorectal cancer | 0.033264 | 21 | 8 |
| GABAergic synapse | 0.037232 | 22 | 13 |
| Endometrial cancer | 0.046372 | 20 | 10 |

TABLE 4B
The miRNAs with relative increases in
AN saliva target metabolic pathways

| KEGG pathway | p-value | #genes | #miRNAs |
|---|---|---|---|
| Steroid hormone biosynthesis | 2.63E−36 | 9 | 1 |
| Drug metabolism - cytochrome P450 | 2.44E−08 | 8 | 1 |
| Ascorbate and aldarate metabolism | 0.001286 | 7 | 1 |
| Chemical carcinogenesis | 0.001286 | 8 | 1 |
| Retinol metabolism | 0.001515 | 9 | 1 |
| Pentose and glucuronate | 0.005547 | 7 | 1 |
| interconversions | | | |
| Starch and sucrose metabolism | 0.005547 | 9 | 2 |
| Porphyrin and chlorophyll | 0.009739 | 7 | 1 |
| metabolism | | | |
| Valine, leucine and | 0.013058 | 2 | 1 |
| isoleucine biosynthesis | | | |
| Drug metabolism - other enzymes | 0.035504 | 7 | 1 |

Together, these five miRNAs demonstrated target enrichment for 9 pathways, 8 of which were related to metabolic/biosynthetic processes. To explore potential relationships between miRNA “alterations” and local expression of coding mRNAs, RNA sequencing data was leveraged through alignment to the human mRNA database. There were 1111 mRNAs with nominal differences in expression between AN, AX, and HC groups. Of these mRNAs, 36 were targets of the 14 miRNAs of interest. The largest number of mRNAs with between-group changes were targeted by hsa-miR-378i (11/36 mRNAs) and hsa-let-7f-2-3p (9/36 mRNAs). There were two mRNAs whose expression was significantly (p<0.05) correlated with miR-378i expression. SLC26A8 was directly associated with miR-378i levels (R=0.32, p=0.015) and OAS3 was inversely related to miR-378i levels (R=−0.29, p=0.023).

The inventors work identified 14 salivary miRNAs with unique expression signatures in adolescent females with AN. MiRNA differences were generally more pronounced in AN/HC comparisons than AN/AX comparisons, suggesting that salivary miRNA levels might reflect both neuropsychiatric and nutritional components of AN. However, the distinct responsivity of individual miRNAs to fasting and post-prandial states in AN patients, coupled with an overwhelming enrichment for biosynthetic gene pathways, means that salivary miRNAs likely play an important role in the metabolic response to caloric intake.

For example, levels of three salivary miRNAs (pre-miR-15b, pre-miR-629, hsa-let-7f-2-3p) demonstrated interactions between AN-status and caloric intake (FIG. 3). All three miRNAs tended to be lower in AN patients during a fasting state, but higher in AN patients in a post-prandial state. Together, these three miRNAs demonstrated significant enrichment for mRNA targets related to fatty acid metabolism (11 genes, 1.03E-17), fatty acid biosynthesis (4 genes, 2.32E-32) and fatty acid degradation (7 genes, p=0.003). Thus, dysregulation of these miRNAs in response to caloric intake may be a hallmark of metabolic disturbance in AN. Indeed, studies of patients with non-alcoholic fatty liver disease have previously demonstrated up-regulation of miR-15b in the serum, which is associated with decreased glucose consumption; Zhang, Y., Cheng, X Lu, Z., Wang, J., Chen, El., Fan, W., . . . & Lu, D. (2013). Upregulation of miR-15b in NAFLD models and in the serum of patients with fatty liver disease. Diabetes research and clinical practice, 99(3), 327-334. In the present study, 9/108 (8%) gene targets for hsa-let-7f-2-3p also showed nominal changes in the saliva of AN patients (Table 5).

TABLE 5
AN-related miRNAs target 36 genes with nominal changes
in “local” oropharyngeal expression

| | | Gene | Context | Gene KW | Gene KW |
|---|---|---|---|---|---|
| MicroRNA | Rank | Target | score | pval | chi-square |
| hsa-miR-378i | 140 | SLC26A8 | −0.305 | 0.047403 | 6.0981 |
| hsa-miR-378i | 9 | OAS3 | −0.341 | 0.017297 | 8.1145 |
| hsa-miR-200b-3p | 49 | CHAF1B | −0.31 | 0.028149 | 7.1405 |
| hsa-miR-30d-5p | 101 | RAB22A | −0.33 | 0.03233 | 6.8635 |
| hsa-let-7f-2-3p | 66 | WT1 | −0.352 | 0.007581 | 9.7643 |
| hsa-miR-30d-5p | 83 | PCMTD2 | −0.451 | 0.041816 | 6.3489 |
| hsa-miR-30d-5p | 105 | TRPM7 | −0.315 | 0.042739 | 6.3053 |
| hsa-let-7f-2-3p | 45 | MTMR12 | −0.311 | 0.033166 | 6.8125 |
| hsa-let-7f-2-3p | 6 | GABRA1 | −0.458 | 0.027498 | 7.1873 |
| hsa-miR-15b-3p | 47 | CSMD3 | −0.326 | 0.042582 | 6.3126 |
| hsa-miR-200b-3p | 60 | PALM2 | −0.343 | 0.038713 | 6.5032 |
| hsa-let-7f-2-3p | 38 | EPN2 | −0.362 | 0.041554 | 6.3615 |
| hsa-miR-200b-3p | 58 | SLC25A27 | −0.455 | 0.042921 | 6.2968 |
| hsa-miR-378i | 340 | ZFAT | −0.437 | 0.039363 | 6.4699 |
| hsa-miR-378i | 182 | SLC9A8 | −0.339 | 0.038256 | 6.5269 |
| hsa-miR-378i | 216 | MYO3B | −0.344 | 0.04038 | 6.4188 |
| hsa-let-7f-2-3p | 62 | GRM1 | −0.33 | 0.01128 | 8.9695 |
| hsa-miR-200c-3p | 59 | PALM2 | −0.343 | 0.038713 | 6.5032 |
| hsa-miR-378i | 187 | FOXG1 | −0.304 | 0.028587 | 7.1096 |
| hsa-miR-378i | 76 | GAB3 | −0.393 | 0.041721 | 6.3535 |
| hsa-miR-200c-3p | 53 | NOC3L | −0.302 | 0.011618 | 8.9103 |
| hsa-miR-203a-3p | 37 | TMEM69 | −0.317 | 0.040904 | 6.3931 |
| hsa-miR-200c-3p | 57 | SLC25A27 | −0.455 | 0.042921 | 6.2968 |
| hsa-miR-30d-5p | 19 | EVX2 | −0.339 | 0.036648 | 6.6128 |
| hsa-miR-30d-5p | 39 | DPY19L1 | −0.318 | 0.04358 | 6.2663 |
| hsa-miR-629-3p | 112 | ARRDC2 | −0.455 | 0.047884 | 6.0779 |
| hsa-let-7f-2-3p | 63 | IQGAP2 | −0.309 | 0.038236 | 6.528 |
| hsa-miR-378i | 339 | ATR | −0.314 | 0.049115 | 6.0272 |
| hsa-miR-378i | 152 | WDR31 | −0.679 | 0.045834 | 6.1655 |
| hsa-miR-378i | 77 | LSM8 | −0.337 | 0.028308 | 7.1292 |
| hsa-let-7f-2-3p | 7 | ERAP1 | −0.347 | 0.039347 | 6.4707 |
| hsa-let-7f-2-3p | 35 | NADK2 | −0.518 | 0.015392 | 8.3477 |
| hsa-miR-378i | 299 | DMRT1 | −0.418 | 0.031203 | 6.9345 |
| hsa-let-7f-2-3p | 27 | SORT1 | −0.33 | 0.040228 | 6.4264 |
| hsa-miR-200b-3p | 54 | NOC3L | −0.302 | 0.011618 | 8.9103 |

Among these genes was SORT1, whose protein product is essential for the adipocyte response to insulin via the SLC2A4/GLUT4 transporter; Li, J., Chen, C Li, Y. Matye, D. J., Wang, Y., Ding, W. X., & Li, T. (2017). Inhibition of insulin/PI3K/AKT signailing decreases adipose Sortilin 1 in mice and 3 T3-L1 adipocytes. Biochimica et Biophysica Acta (BBA)-Molecular Basis of Disease, 1863(11), 2924-2933.

Intriguingly, hsa-let-7f-2-3p also targets GABRA1 and GRM1, elements of the GABA signaling pathway, whose transcripts are also “disrupted” in saliva of AN patients relative to AX and HC groups (Table 5). In fact, 13/14 miRNAs of interest target a gene in the GABA signaling pathway (Table 4A). Given the importance of functional GABA signaling in the brain's food-reward system (see Wu, Q., R. Palmiter, R. D. (2011). GABAergic signaling by AgRP neurons prevents anorexia via a melanocortin-independent mechanism. European journal of pharmacology, 660(1), 21-27), and the association of GABA-related single nucleotide polymorphisms with AN, hsa-let-7f-2-3p may be a critical molecule linking the neuropsychiatric and metabolic characteristics of AN. This idea is supported by the association between post-prandial levels of hsa-let-7f-2-3p and GAD7 scores in AN participants.

A number of other miRNAs of interest also demonstrated correlations with neuropsychiatric measures in AN patients. For instance, hsa-miR-30d-5p and hsa-miR-374a-3p were associated with GAD7 scores, while pre-miR-15b was associated with CBCL scores and pre-miR-103b-1 was associated with EDEQ measures (Table 3). Neurotransmitter influences, such as GABA are one explanation for these associations. However, three of these miRNAs (hsa-miR-374a-3p, pre-miR-15b, pre-miR-103b-1) were also associated with TSH/T3 levels in the serum of AN patients. This finding provides another example of how salivary miRNAs may serve to link neuropsychiatric and metabolic disruptions in eating disorders.

The strongest miRNA/clinical association was observed between salivary levels of pre-miR-200c and eating disorder duration. Notably, pre-miR-200c also had the largest difference in AN patients, and the largest VIP score on group PLSDA. Levels of pre-miR-200c were generally up-regulated in AN patients relative to AX and HC groups. This is consistent with studies in animal models of non-learned helplessness which have shown altered miR-200c responses in association with depression-like symptoms; Dwivedi, Y. (2013). microRNAs as biomarker in depression pathogenesis. Annals of psychiatry and mental health, 1(1). 1003. In addition, increased levels of miR-200c have been linked to reactive oxygen species in a study of diabetic cardiomyocytes exposed to extreme glucose fluctuations. Thus, this miRNA may potentially serve as a hallmark of protracted malnutrition and a warning sign for AN-related cardiovascular complications.

Further studies examining the longitudinal expression of peripheral miRNAs over the course of AN therapy are certainly warranted. Such studies may provide valuable markers to track the physiologic response to therapy, or predict duration/extent of recovery at the time of admission to a therapy program. In addition, miRNA signatures may prove useful for sub-categorizing groups of eating disorder patients by predominance of restricting, over-exercising, self-induced vomiting, or other symptoms. This preliminary study suggests that either fasting, or post-prandial salivary samples may be used for monitoring miRNA levels in AN patients. However, given the responsiveness of many miRNAs to caloric intake, a random mix of these techniques, without controlling for the time of last meal is likely to confound results.

Non-limiting embodiments of this invention or technology include the following:

1. A method for detecting a risk of, diagnosing, or monitoring anorexia nervosa (“AN”) or another eating disorder comprising:

detecting at least one abnormal or altered pattern of miRNA in biological sample compared to a control value from one or more normal subjects or healthy controls, and

selecting a subject having at least one abnormal or altered pattern of miRNA correlated with anorexia nervosa or another eating disorder.

2. The method of embodiment 1, further comprising conducting a subjective assessment of the subject to establish a DSM-V diagnosis; or determining whether the subject has improved prognosis or fewer symptoms or prognosing a chronic course of the AN or eating disorder.

3. The method of embodiment 1, further comprising administering a treatment, modifying a treatment course, or terminating a treatment for anorexia or other eating disorder.

4. The method of embodiment 1, wherein the eating disorder is anorexia nervosa.

5. The method of embodiment 1, wherein the abnormal or altered pattern is associated with depression, anxiety, a sleep disorder, or other comorbidity.

6. The method of embodiment 1, wherein said abnormal or altered pattern identifies a subject most likely to be healthy, to have anorexia nervosa (“AN”), or to have an anxiety disorder (“AX”).

7. The method of embodiment 1, wherein the biological sample is saliva.

8. The method of embodiment 1, wherein the biological sample is serum.

9. The method of embodiment 1 that comprises detecting at least one abnormal or altered quantity of miRNA.

10. The method of embodiment 1 that comprises detecting at least one abnormal or altered quantity of microbial RNA.

11. The method of embodiment 1 that comprises detecting at least one abnormal or altered pattern of miRNA.

12. The method of embodiment 1, wherein the subject is female of an age of no more than >0, 1, 2, 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95 or ≥100 years (or any intermediate age value).

13. The method of embodiment 1, wherein the subject is an adolescent female.

14. The method of embodiment 1 that comprises monitoring a subject having anorexia nervosa or another eating disorder by detecting at least one abnormal or altered pattern of miRNA and/or microbial RNA more than once; optionally selecting a subject with exacerbation or remission of anorexia nervosa or other eating disorder when levels of miRNAs or microbial RNAs associated with anorexia nervosa or another eating disorder are trending away from or towards a control value; and optionally treating the subject, modifying treatment of the subject, or diminishing or terminating treatment of the subject.

15. The method of embodiment 1, wherein the biological sample is a fasting or pre-prandial sample; and optionally where one or more subsequent miRNA or microbial RNA levels are compared to or normalized to the fasting or pre-prandial value taken.

16. The method of embodiment 1, wherein the biological sample is a post-prandial sample taken immediately after eating, or 15 mins, 30 mins, 45 mins, 60 mins, 120 mins, or 240 mins (or any intermediate time) after eating; and optionally where one or more subsequent miRNA or microbial RNA levels are compared to or normalized to the post-prandial value taken.

17. The method of embodiment 1, wherein the abnormal or altered pattern is detected in an amount of one or more miRNAs.

18. The method of embodiment 1, wherein the abnormal or altered pattern is detected in an amount of one or more miRNAs, wherein said miRNAs comprise at least one of hsa-miR-378i, pre-miR-145, and pre-miR-744; and/or at least one of hsa-miR-203a-3p, pre-miR-200c, and hsa-miR-200b-3p).

19. The method of embodiment 1, wherein the abnormal or altered pattern is detected in an amount of one or more miRNAs, wherein said miRNAs comprise at least one of pre-miR-15b, hsa-let-7f-2-3p, and pre-miR-629.

20. The method of embodiment 1, wherein the abnormal or altered pattern in an amount of one or more miRNAs is detected, wherein said miRNAs comprise at least one of pre-mir-200c, hsa-miR-30d-5p, has-miR-374a-3p, pre-mir-30a, and has-miR-200b-3p. (Table 1)

21. The method of embodiment 1, wherein the abnormal or altered pattern in an amount of one or more miRNAs is detected and wherein said miRNAs comprise at least one of hsa-let-7a-5p, hsa-miR-200b-3p, hsa-let-7f-2-3p, pre-mir-629, pre-mir-98, pre-mir-15b, hsa-miR-629-5p, hsa-miR-6073, hsa-miR-197-3p, pre-mir-197, pre-mir-29a, hsa-miR-203a-3p, hsa-miR-106a-5p, pre-mir-103b-1 and pre-mir-374c (Table 2).

22. The method of embodiment 1, wherein selecting excludes or normalizes to its pre-meal concentration value a concentration of at least one of hsa-let-7a-5p, hsa-miR-200b-3p, hsa-let-7f-2-3p, pre-mir-629, pre-mir-98, pre-mir-15b, hsa-miR-629-5p, hsa-miR-6073, hsa-miR-19′7-3p, pre-mir-197, pre-mir-29a, hsa-miR-203a-3p, hsa-miR-106a-5p, pre-mir-103b-1 and pre-mir-374c (Table 2).

23. The method of embodiment 1 comprising:

detecting a meal-induced change in at least one salivary miRNA level selected from the group consisting of Pre-miR-200c, Hsa-miR-30d-5p, Hsa-miR-374a-3p, Pre-miR-30a, Hsa-miR-200b-3p, Pre-miR-15b, Hsa-let-7f-2-3p, Pre-miR-629, Hsa-miR-378i, Hsa-miR-200c-3p, Pre-miR-145, Hsa-miR-203a-3p, Pre-miR-103b-1 and Pre-miR-744 (Table 3) and

selecting a subject having a medical feature associated with said meal-induced change in miRNA levels (such as a medical feature described by Table 3).

24. The method of embodiment 1, wherein the at least one miRNA targets a gene involved in a metabolic or biosynthetic pathway (such as a KEGG pathway described by Table 4A).

25. The method of embodiment 1, wherein the at least one miRNA exhibits a relative increase in anorexia target metabolic pathway (such as a KEGG pathway described by Table 4B).

26. The method of embodiment 1, wherein the at least one miRNA target at least one gene selected from the group consisting of SLC26A8, OAS3, CHAF1B, RAB22A, WT1, PCMTD2, TRPM7, MTMR12, GABRA1, CSMD3, PALM2, EPN2, SLC25A27, ZFAT, SLC9A8, MYO3B, GRM1, PALM2, FOXG1, GAB3, NOC3L, TMEM69, SLC25A27, EVX2, DPY19L1, ARRDC2, IQGAP2, ATR, WDR31, LSM8, ERAP1, NADK2, DMRT1, SORT1, and NOC3L (Table 5); or

wherein said at least one miRNA is selected from the group consisting of hsa-miR-378i, hsa-miR-378i, hsa-miR-200b-3p, hsa-miR-30d-5p, hsa-let-7f-2-3p, hsa-miR-30d-5p, hsa-miR-30d-5p, hsa-let-7f-2-3p, hsa-let-7f-2-3p, hsa-miR-15b-3p, hsa-miR-200b-3p, hsa-let-7f-2-3p, hsa-miR-200b-3p, hsa-miR-378i, hsa-miR-378i, hsa-miR-378i, hsa-let-7f-2-3p, hsa-miR-200c-3p, hsa-miR-378i, hsa-miR-378i, hsa-miR-200c-3p, hsa-miR-203a-3p, hsa-miR-200c-3p, hsa-miR-30d-5p, hsa-miR-30d-5p, hsa-miR-629-3p, hsa-let-7f-2-3p, hsa-miR-378i, hsa-miR-378i, hsa-miR-378i, hsa-let-7f-2-3p, hsa-let-7f-2-3p, hsa-miR-378i, hsa-let-7f-2-3p, and hsa-miR-200b-3p. (Table 5).

27. The method of embodiment 1 comprising detecting depression of a level of at least one of pre-miR-15b, hsa-let-7f-2-3p and pre-miR-629 in a fasting saliva sample compared to a healthy control and/or an elevation of at least one of pre-miR-15b, hsa-let-7f-2-3p and pre-miR-629 in a post-prandial saliva sample compared to a healthy control (FIG. 3), and

selecting a subject having AN or at risk of AN when said fasting saliva sample has an depressed amount of said at least one miRNA and/or when a post-prandial saliva sample has an elevated amount of said at least one miRNA.

28. The method of embodiment 1 for determining or estimating a duration of an eating disorder comprising determining the level of pre-miR-200c compared to a healthy control, wherein a duration of the eating disorder is inversely proportional to the amount of pre-miR-200c detected.

29. The method of embodiment 1 for determining, estimating, or monitoring an estradiol level comprising determining a level of at least one of pre-miR-200c, hsa-miR-200b, pre-miR-15b, or pre-miR-145 compared to a healthy control.

30. The method of embodiment 1 for determining, estimating or monitoring a level of thyroid stimulating hormone (“TSH”) comprising determining a level of at least one of hsa-miR-103b, hsa-miR-378i, or miR-200c compared to a healthy control.

31. A composition having two or more probes that detect miRNAs or microbial RNAs associated with anorexia nervosa or another eating disorder.

32. A kit for detection of miRNAs or microbial RNAs in saliva comprising 1, 2, 3, 4, 5, 6, 7, 8, 9, 10, 20, 50 or more probes or primers that recognize miRNAs or microbial RNAs associated with anorexia nervosa or another eating disorder, and optionally, excipients, buffers, platforms, containers, indicators, packing materials or instructions for use.

33. The kit of embodiment 31, wherein the one or more probes or primers recognize miRNAs or microRNAs associated with anorexia nervosa or another eating disorder.

34. The kit of embodiment 31, wherein the one or more probes or primers recognize at least one miRNA selected from the group consisting of pre-mir-200c, hsa-miR-30d-5p, hsa-miR-374a-3p, pre-mir-30a, and hsa-miR-200b-3p (Table 1); consisting of hsa-let-7a-5p, hsa-miR-200b-3p, hsa-let-7f-2-3p, pre-mir-629, pre-mir-98, pre-mir-15b, hsa-miR-629-5p, hsa-miR-6073, hsa-miR-197-3p, pre-mir-197, pre-mir-29a, hsa-miR-203a-3p, hsa-miR-106a-5p, pre-mir-103b-1 and pre-mir-374c (Table 2); and/or consisting of Pre-miR-200c, Hsa-miR-30d-5p, Hsa-miR-374a-3p, Pre-miR-30a, Hsa-miR-200b-3p, Pre-miR-15b, Hsa-let-7f-2-3p, Pre-miR-629, Hsa-miR-378i, Hsa-miR-200c-3p, Pre-miR-145, Hsa-miR-203a-3p, Pre-miR-103b-1 and Pre-miR-744 (Table 3).

35. The kit of embodiment 31, wherein the one or more probes or primers recognize at least one miRNA that targets a gene involved in a metabolic or biosynthetic pathway (such as a KEGG pathway described by Table 4A).

36. The kit of embodiment 31, wherein the probe or primer recognize at least one miRNA that exhibits a relative increase in anorexia target metabolic pathway (such as a KEGG pathway described by Table 4B).

37. The kit of embodiment 31, wherein the at least one probe or primer recognizes a miRNA that targets at least one gene selected from the group consisting of SLC26A8, OAS3, CHAF1B, RAB22A, WT1, PCMTD2, TRPM7, MTMR12, GABRA1, CSMD3, PALM2, EPN2, SLC25A27, ZFAT, SLC9A8, MYO3B, GRM1, PALM2, FOXG1, GAB3, NOC3L, TMEM69, SLC25A27, EVX2, DPY19L1, ARRDC2, IQGAP2, ATR, WDR31, LSM8, ERAP1, NADK2, DMRT1, SORT1, and NOC3L (Table 5) or

wherein said at least one probe or primer recognizes miRNA selected from the group consisting of. hsa-miR-378i, hsa-miR-378i, hsa-miR-200b-3p, hsa-miR-30d-5p, hsa-let-7f-2-3p, hsa-miR-30d-5p, hsa-miR-30d-5p, hsa-let-7f-2-3p, hsa-let-7f-2-3p, hsa-miR-15b-3p, hsa-miR-200b-3p, hsa-let-7f-2-3p, hsa-miR-200b-3p, hsa-miR-378i, hsa-miR-378i, hsa-miR-378i, hsa-let-7f-2-3p, hsa-miR-200c-3p, hsa-miR-378i, hsa-miR-378i, hsa-miR-200c-3p, hsa-miR-203a-3p, hsa-miR-200c-3p, hsa-miR-30d-5p, hsa-miR-30d-5p, hsa-miR-629-3p, hsa-let-7f-2-3p, hsa-miR-378i, hsa-miR-378i, hsa-miR-378i, hsa-let-7f-2-3p, hsa-let-7f-2-3p, hsa-miR-378i, hsa-let-7f-2-3p, and hsa-miR-200b-3p (Table 5).

38. A microarray comprising a set of probes comprising nucleotide sequences that can detect and quantify at least one miRNA sequence and/or microbial RNA sequence associated with anorexia nervosa or another eating disorder.

39. The microarray of embodiment 37, further comprising a set of at least 10, preferably at least 15, more preferably at least 20 probes comprising nucleotide sequences corresponding to sequences of miRNAs and/or microbial RNAs associated with associated with anorexia nervosa or another eating disorder.

40. A method of treating a subject having anorexia nervosa or another eating disorder comprising treating the subject with one or more of drug therapy, antimicrobial therapy, medical regimen, a diet therapy, psychotherapy, a behavior therapy, a communication therapy or an alternative medical therapy, wherein the subject was identified as having symptoms of AN or eating disorder by the method of embodiment 1.

41. A method for assessing or monitoring nutritional status of a subject comprising detecting at least one abnormal or altered pattern of miRNA in saliva sample compared to a control value from one or more normal subjects or healthy controls, and selecting a subject having at least one abnormal or altered pattern of miRNA correlated with anorexia, chronic malnutrition, or other nutritional deficit.

42. The method of embodiment 40, further comprising administering at least one treatment for anorexia, chronic malnutrition or other nutritional deficit to said subject.

43. The method of embodiment 40, wherein at least one of pre-mir-200c, hsa-miR-200b-3p, hsa-miR-378i, hsa-miR-200c-3p, pre-miR-145, hsa-miR-203a-3p, pre-miR-103b-1, pre-miR-744, hsa-miR-30d-5p, hsa-miR-374a-3p, or pre-mir-30a is detected.

44. The method of embodiment 40, wherein the subject is cachexic.

45. The method of embodiment 40, wherein the subject has or exhibits cancer, AIDS, hepatitis, coeliac disease, chronic obstructive pulmonary disease, helminth or other parasite infection, drug abuse or addiction, alcohol abuse or addiction, multiple sclerosis, rheumatoid arthritis, congestive heart failure, tuberculosis, familial amyloid polyneuropathy, mercury or other heavy metal poisoning (acrodynia), Crohn's disease, type 1 diabetes mellitus, type 2 diabetes, anorexia nervosa, hormonal imbalance or deficiency, unexplained weight loss, or is a pre-surgical or post-surgical patient.

46. A method for assessing or monitoring meal or nutritional-intake status of a subject comprising:

detecting at least one abnormal or altered pattern of miRNA in a saliva sample compared to a control value, and

selecting a subject having at least one variation or altered pattern of miRNA concentrations associated with post-meal status; wherein post-meal status may indicate intake of nutrients within the last 1, 2, 5, 10, 20, 30, 40, 50 or <60 mins or in the last 1, 2, 3, 4, 5, 6, 8, 10 or 12 hours (or any intermediate value within this range) having at least one abnormal or altered pattern of miRNA correlated with anorexia, chronic malnutrition, or other nutritional deficit.

47. The method of embodiment 45, wherein at least one of hsa-let-7a-5p, hsa-miR-200b-3p, hsa-let-7f-2-3p, pre-mir-629, pre-mir-98, pre-mir-15b, hsa-miR-629-5p, hsa-miR-6073, hsa-miR-197-3p, pre-mir-197, pre-mir-29a, hsa-miR-203a-3p, hsa-miR-106a-5p, pre-mir-103b-1 or pre-mir-374c is detected.

48. The method of embodiment 45, wherein at least one of hsa-let-7a-5p, hsa-miR-200b-3p, pre-mir-29a, hsa-miR-203a-3p, pre-mir-103b-1 or pre-mir-374c is detected.

49. The method of embodiment 45, wherein a subject having oral intake of at least one nutrient is selected.

50. The method of embodiment 45, wherein a subject having parenteral intake of at least one nutrient is selected.

51. The method of embodiment 45, further comprising administering at least one treatment for anorexia, chronic malnutrition or other nutritional deficit to said subject.

52. A method for diagnosing anorexia or risk of anorexia comprising:

detecting at least one abnormal or altered pattern of miRNA in a saliva sample compared to a control value, wherein said miRNA is at least one of hsa-let-7a-5p, hsa-miR-200b-3p, pre-mir-29a, hsa-miR-203a-3p, pre-mir-103b-1 or pre-mir-374c, and

selecting a subject at risk of or having anorexia when an abnormal or altered pattern of said miRNA(s) is detected compared to a control value from a healthy, nonanorexic subject; and optionally further evaluating the subject for anorexia or administering a treatment for anorexia.

53. A method for diagnosing anorexia or risk of anorexia comprising:

detecting at least one abnormal or altered pattern of miRNA in a saliva sample compared to a control value, wherein said miRNA is at least one of hsa-let-7f-2-3p, pre-mir-629, or pre-mir-15b, and

selecting a subject at risk of or having anorexia when an abnormal or altered pattern of said miRNA(s) is detected compared to a control value from a healthy, non-anorexic subject; and optionally further evaluating the subject for anorexia or administering a treatment for anorexia.

The materials, methods, and examples are illustrative only and are not intended to be limiting, unless otherwise specified. Numerous modification and variations on the present invention are possible in light of the above teachings. It is therefore to be understood that within the scope of the appended claims, the invention may be practiced otherwise than as specifically described herein.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

### REFERENCES

1. Erskine, H. E., Whiteford, H. A. & Pike, K. M. The global burden of eating disorders. Current opinion in psychiatry, doi:10.1097/YCO.0000000000000276 (2016).
2 Kaye, W. H., Wierenga, C. E., Bailer, U. F., Simmons, A. N. & Bischoff-Grethe, A. Nothing tastes as good as skinny feels: the neurobiology of anorexia nervosa. Trends in neurosciences 36, 110-120, doi:10.1016/j.tins.2013.01.003 (2013).
3 Kaye, W. H., Fudge, J. L. & Paulus, M. New insights into symptoms and neurocircuit function of anorexia nervosa. Nature reviews. Neuroscience 10, 573-584, doi:10.1038/nrn2682 (2009).
4 Mairs, R. & Nicholls, D. Assessment and treatment of eating disorders in children and adolescents. Archives of disease in childhood, doi:10.1136/archdischild-2015-309481 (2016).
5 Steinhausen, H. C. Outcome of eating disorders. Child and adolescent psychiatric clinics of North America 18, 225-242, doi:10.1016/j.chc.2008.07.013 (2009).
6 Spalding A L, F. S., Ornstein R M, Mammel K A, Woods E R, Richmond T K. Determining the feasibility of a national prospective eating disorder registry: a qualitative study. J Adol Health 58, S38 (2016).
7 Bocchio-Chiavetto, L. et al. Blood microRNA changes in depressed patients during antidepressant treatment. European neuropsychopharmacology: the journal of the European College of Neuropsychopharmacology 23, 602-611, doi:10.1016/j.euroneuro.2012.06.013 (2013).
8 Mercader, J. M. et al. Aberrant brain microRNA target and miRISC gene expression in the anx/anx anorexia mouse model. Gene 497, 181-190, doi:10.1016/j.gene.2012.01.057 (2012).
9 Kolshus, E., Dalton, V. S., Ryan, K. M. & McLoughlin, D. M. When less is more—microRNAs and psychiatric disorders. Acta psychiatrica Scandinavica 129, 241-256, doi:10.1111/acps.12191 (2014).
10 Smalheiser, N. R. et al. MicroRNA expression is down-regulated and reorganized in prefrontal cortex of depressed suicide subjects. PloS one 7, e33201, doi:10.1371/journal.pone.0033201 (2012).
11 Belzeaux, R. et al. Responder and nonresponder patients exhibit different peripheral transcriptional signatures during major depressive episode. Translational psychiatry 2, e185, doi:10.1038/tp.2012.112 (2012).
12 Hicks, S. D., Ignacio, C., Gentile, K. & Middleton, F. A. Salivary miRNA profiles identify children with autism spectrum disorder, correlate with adaptive behavior, and implicate ASD candidate genes involved in neurodevelopment. BMC pediatrics 16, 52, doi:10.1186/s12887-016-0586-x (2016).
13 Monteleone, P. et al. Asymmetry of salivary cortisol and alpha-amylase responses to psychosocial stress in anorexia nervosa but not in bulimia nervosa. Psychological medicine 41, 1963-1969, doi:10.1017/50033291711000092 (2011).

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A method for detecting a risk of, diagnosing, or monitoring anorexia nervosa (“AN”) or another eating disorder comprising:
detecting at least one abnormal or altered pattern of miRNA in a biological sample compared to a control value from one or more normal subjects or healthy controls, and
selecting a subject having at least one abnormal or altered pattern of miRNA correlated with anorexia nervosa or another eating disorder; and
treating the selected subject for anorexia nervosa or another eating disorder.

2-16. (canceled)

17. The method of claim 1, wherein the biological sample is saliva and where the abnormal or altered pattern is detected in an amount of one or more salivary miRNAs.

18. The method of claim 1, wherein the abnormal or altered pattern is detected in an amount of one or more miRNAs, wherein said miRNAs comprise at least one of hsa-miR-378i, pre-miR-145, or pre-miR-744; and/or at least one of hsa-miR-203a-3p, pre-miR-200c, or and hsa-miR-200b-3p).

19. The method of claim 1, wherein the abnormal or altered pattern is detected in an amount of one or more miRNAs, wherein said miRNAs comprise at least one of pre-miR-15b, hsa-let-7f-2-3p, or pre-miR-629.

20. The method of claim 1, wherein the abnormal or altered pattern in an amount of one or more miRNAs is detected, wherein said miRNAs comprise at least one of pre-mir-200c, hsa-miR-30d-5p, has-miR-374a-3p, pre-mir-30a, or and has-miR-200b-3p.

21. The method of claim 1, wherein the abnormal or altered pattern in an amount of one or more miRNAs is detected and wherein said miRNAs comprise at least one of hsa-let-7a-5p, hsa-miR-200b-3p, hsa-let-7f-2-3p, pre-mir-629, pre-mir-98, pre-mir-15b, hsa-miR-629-5p, hsa-miR-6073, hsa-miR-197-3p, pre-mir-197, pre-mir-29a, hsa-miR-203a-3p, hsa-miR-106a-5p, pre-mir-103b-1 or and pre-mir-374c.

22. The method of claim 1, wherein selecting excludes or normalizes to its pre-meal concentration value a concentration of at least one of hsa-let-7a-5p, hsa-miR-200b-3p, hsa-let-7f-2-3p, pre-mir-629, pre-mir-98, pre-mir-15b, hsa-miR-629-5p, hsa-miR-6073, hsa-miR-197-3p, pre-mir-197, pre-mir-29a, hsa-miR-203a-3p, hsa-miR-106a-5p, pre-mir-103b-1 or and pre-mir-374c.

23. The method of claim 1 comprising:
detecting a meal-induced change in at least one salivary miRNA level selected from the group consisting of Pre-miR-200c, Hsa-miR-30d-5p, Hsa-miR-374a-3p, Pre-miR-30a, Hsa-miR-200b-3p, Pre-miR-15b, Hsa-let-7f-2-3p, Pre-miR-629, Hsa-miR-378i, Hsa-miR-200c-3p, Pre-miR-145, Hsa-miR-203a-3p, Pre-miR-103b-1 and Pre-miR-744 and
selecting a subject having a medical feature associated with said meal-induced change in miRNA levels.

24.-25. (canceled)

26. The method of claim 1, wherein the at least one abnormal or altered pattern of miRNA comprises miRNA targeting at least one miRNA target at least one gene selected from the group consisting of SLC26A8, OAS3, CHAF1B, RAB22A, WT1, PCMTD2, TRPM7, MTMR12, GABRA1, CSMD3, PALM2, EPN2, SLC25A27, ZFAT, SLC9A8, MYO3B, GRM1, PALM2, FOXG1, GAB3, NOC3L, T1VIEM69, SLC25A27, EVX2, DPY19L1, ARRDC2, IQGAP2, ATR, WDR31, LSM8, ERAP1, NADK2, DMRT1, SORT1, and NOC3L; or wherein said at least one abnormal or altered pattern of miRNA comprises at least one miRNA selected from the group consisting of hsa-miR-378i, hsa-miR-378i, hsa-miR-200b-3p, hsa-miR-30d-5p, hsa-let-7f-2-3p, hsa-miR-30d-5p, hsa-miR-30d-5p, hsa-let-7f-2-3p, hsa-let-7f-2-3p, hsa-miR-15b-3p, hsa-miR-200b-3p, hsa-let-7f-2-3p, hsa-miR-200b-3p, hsa-miR-378i, hsa-miR-378i, hsa-miR-378i, hsa-let-7f-2-3p, hsa-miR-200c-3p, hsa-miR-378i, hsa-miR-378i, hsa-miR-200c-3p, hsa-miR-203a-3p, hsa-miR-200c-3p, hsa-miR-30d-5p, hsa-miR-30d-5p, hsa-miR-629-3p, hsa-let-7f-2-3p, hsa-miR-378i, hsa-miR-378i, hsa-miR-378i, hsa-let-7f-2-3p, hsa-let-7f-2-3p, hsa-miR-378i, hsa-let-7f-2-3p, and hsa-miR-200b-3p.

27. The method of claim 1 comprising
detecting depression of a level of at least one of pre-miR-15b, hsa-let-7f-2-3p or pre-miR-629 in a fasting saliva sample compared to a healthy control and/or an elevation of at least one of pre-miR-15b, hsa-let-7f-2-3p or pre-miR-629 in a post-prandial saliva sample compared to a healthy control, and
selecting a subject having AN or at risk of AN when said fasting saliva sample has an depressed amount of said at least one miRNA and/or when a post-prandial saliva sample has an elevated amount of said at least one miRNA.

28. The method of claim 1 for determining or estimating a duration of an eating disorder comprising determining the level of pre-miR-200c compared to a healthy control, and
detecting or estimating the duration of the eating disorder based on the amount of pre-miR-200c detected compared to a healthy control value, wherein the duration of the eating disorder is inversely proportional to the amount of pre-miR-200c detected.

29. The method of claim 1, further comprising determining, estimating, or monitoring an estradiol level comprising determining an abnormal or altered level of at least one of pre-miR-200c, hsa-miR-200b, pre-miR-15b, or pre-miR-145 compared to a healthy control.

30. The method of claim 1, further comprising determining, estimating or monitoring a level of thyroid stimulating hormone (“TSH”) comprising determining an abnormal or altered level of at least one of hsa-miR-103b, hsa-miR-378i, or miR-200c compared to a healthy control.

31.-39. (canceled)

40. The method of claim 1, further comprising treating the subject with one or more of drug therapy, antimicrobial therapy, medical regimen, a diet therapy, psychotherapy, a behavior therapy, a communication therapy or an alternative medical therapy, wherein the subject was identified as having symptoms of AN or an eating disorder.

41. A method for assessing or monitoring nutritional status of a subject comprising detecting at least one abnormal or altered pattern of miRNA in saliva sample compared to a control value from one or more normal subjects or healthy controls, and
selecting a subject having at least one abnormal or altered pattern of miRNA correlated with anorexia, chronic malnutrition, or other nutritional deficit; and
treating the selected subject for anorexia, chronic malnutrition or other nutritional deficit.

42. The method of claim 41, further comprising administering at least one treatment for anorexia, chronic malnutrition or other nutritional deficit to said subject.

43. The method of claim 41, wherein at least one of pre-mir-200c, hsa-miR-200b-3p, hsa-miR-378i, hsa-miR-200c-3p, pre-miR-145, hsa-miR-203a-3p, pre-miR-103b-1, pre-miR-744, hsa-miR-30d-5p, hsa-miR-374a-3p, or pre-mir-30a is detected.

44-45. (canceled)

46. A method for assessing or monitoring meal or nutritional-intake status of a subject comprising:
detecting at least one abnormal or altered pattern of miRNA in a saliva sample compared to a control value,
selecting a subject having at least one variation or altered pattern of miRNA concentrations associated with post-meal status compared to pre-meal status correlated with anorexia, chronic malnutrition, or other nutritional deficit, and
treating the selected subject for anorexia, chronic malnutrition or other nutritional deficit.

47. The method of claim 46, wherein at least one of hsa-let-7a-5p, hsa-miR-200b-3p, hsa-let-7f-2-3p, pre-mir-629, pre-mir-98, pre-mir-15b, hsa-miR-629-5p, hsa-miR-6073, hsa-miR-197-3p, pre-mir-197, pre-mir-29a, hsa-miR-203a-3p, hsa-miR-106a-5p, pre-mir-103b-1 or pre-mir-374c is detected.

48.-51. (canceled)

52. The method of claim 1, wherein said subject is in need of diagnosing anorexia or risk of anorexia comprising:
detecting at least one abnormal or altered pattern of miRNA in a saliva sample compared to a control value, wherein said miRNA is at least one of hsa-let-7a-5p, hsa-miR-200b-3p, pre-mir-29a, hsa-miR-203a-3p, pre-mir-103b-1 or pre-mir-374c, or wherein said miRNA is at least one of hsa-let-7f-2-3p, pre-mir-629, or pre-mir-15b; and
selecting a subject at risk of or having anorexia when an abnormal or altered pattern of said miRNA(s) is detected compared to a control value from a healthy, nonanorexic subject; and optionally further evaluating the subject for anorexia or administering a treatment for anorexia.

53. (canceled)
